(19) United States
(12) Patent Application Publication
(10) Pub. No.: US 2013/0004522 A1
(43) Pub. Date: Jan. 3, 2013

(54) PHOTOTRIGGERED NANOPARTICLES FOR CELL AND TISSUE TARGETING

(76) Inventors: Tal Dvir, Rishon le Zion (IL); Daniel S. Kohane, Newton, MA (US); Matthew Ryan Banghart, Boston, MA (US); Robert S. Langer, Newton, MA (US)

(21) Appl. No.: 13/499,543

(22) PCT Filed: Sep. 30, 2010
(86) PCT No.: PCT/US10/50846

§ 371 Date: Sep. 13, 2012

Related U.S. Application Data
(60) Provisional application No. 61/247,535, filed on Sep. 30, 2009.

(51) Int. Cl. A61K 38/08; C07K 17/02; A61K 39/395

(52) U.S. Cl. 424/178.1; 514/21.6; 530/391.7; 530/328

(57) ABSTRACT

The present invention relates, in part, to a novel and simple particulate system that targets and binds any tissue selectively upon light illumination. The particulate system can be used for targeted delivery of substances to predefined cells or tissues in an individual.

### RELATED APPLICATIONS

This application claims the benefit of the filing date of U.S. provisional patent application 61/247,535, filed Sep. 30, 2009 entitled “Phototriggered nanoparticles for cell and tissue targeting”. The entire teachings and contents of the referenced provisional application is incorporated herein by reference.

### FEDERALLY SPONSORED RESEARCH

The invention was supported, in whole or in part, by grant No. GM073626 from the National Institute of Health (NIH). The Government has certain rights in the invention.

### FIELD OF THE INVENTION

The present invention relates to compositions and methods for targeted delivery of substances in an individual.

### BACKGROUND OF THE INVENTION

A major setback associated with drug therapy is the inability to carry therapeutic agents to a specific site of the body without causing nonspecific toxicity or inefficient therapy. With advances in nanotechnology, a central focus in drug delivery research is given to developing techniques for modifying surfaces of nanoparticles with targeting moieties which allow them to specifically recognize and bind to unique properties of diseased cells and tissues and thus, to increase targeting efficiency. Such targeters are usually composed of antibodies, peptides or aptamers and their binding sites on cells are specific receptors, channels or other molecules present on the cell membrane.

Recent studies have successfully demonstrated selective targeting of engineered nanoparticles to tumors and the feasibility of such targeting systems has already been clinically demonstrated. In order to engineer such targeting systems, the nanoparticulate system is more effective if it overcomes two main barriers on the pathway between the circulatory and the target cells. The first hurdle is the inefficient ability of the nanocarriers to leave the vascular system by penetrating between the endothelial cells comprising the blood vessels. In targeting systems designed for the treatment of cancer, researchers rely on the leaky blood vessels in the diseased area, which allow easy penetration of the nanoparticles and infiltration toward the diseased cells. The second hurdle is finding a unique expression of membrane proteins on the diseased cells and designing a specific ligand that can serve as a targeter. Since many diseases do not provide to researchers the luxury of having leaky blood vessels where the nanoparticles can easily exit the circulation, or the cells do not possess known unique biomarkers that can serve as targets, there is an urgent need to find and investigate new approaches to target therapeutic agent-loaded nanoparticles towards diseased tissues and organs.

### SUMMARY OF THE INVENTION

The invention relates, in one aspect, to the discovery of compositions for delivering agents/substances to a target site by providing a composition that includes a delivery moiety attached to a targeting moiety. Accordingly, one aspect of the invention involves compositions comprising a plurality of particles, each particle containing an effective amount of a substance to be delivered to an individual, wherein a targeting ligand inactivated by caging using a photo-removable protecting group is attached to the surface of the particles, wherein the inactive ligand is activated by removal of the protecting group by irradiation of the composition, and wherein the active ligand is capable of binding an anti-ligand.

According to some aspects of the invention, methods for targeted delivery of a substance to predefined cells or tissues in an individual are provided. In some embodiments, the methods comprise administering to an individual in need thereof a composition comprising particles containing an effective amount of a substance to be delivered to the individual, wherein a targeting ligand inactivated by caging using a photo-removable protecting group is attached to the surface of the particles, and selectively irradiating predefined cells or tissues in the individual to activate the inactive ligand in the irradiated predefined cells or tissues by removal of the protecting group, wherein the active ligand is capable of binding along with the attached particles to an anti-ligand present on the predefined cells or tissues leading to the targeted delivery of the substance to the individual.

According to some aspects of the invention, methods for targeted delivery of a substance to predefined cells or tissues in an individual are provided. In some embodiments, the methods comprise administering to an individual in need thereof a composition comprising particles containing an effective amount of a substance to be delivered to the individual, wherein a targeting peptide inactivated by caging using a photo-removable protecting group is attached to the surface of the particles; and selectively irradiating predefined cells or tissues in the individual to activate the inactive peptide in the irradiated predefined cells or tissues by removal of the protecting group, wherein the active peptide is capable of binding along with the attached particles to integrins present on the predefined cells or tissues leading to the targeted delivery of the substance to the individual.

According to some aspects of the invention, compositions comprising a plurality of particles are provided. In some embodiments, each particle is capable of carrying an effective amount of a substance to be delivered to an individual, wherein a targeting ligand inactivated by caging using a photo-removable protecting group is attached to the surface of the particles, wherein the inactive ligand is activated by removal of the protecting group by irradiation of the composition, and wherein the active ligand is capable of binding an anti-ligand.

The following embodiments apply equally to the various aspects of the invention set forth herein unless indicated otherwise.

In some embodiments, the ligand comprises peptides, antibodies, and/or aptamers. In some embodiments, the petides comprise a RGD or YIGSR (SEQ ID NO: 1) amino acid motif. In some embodiments, the photo-removable protecting group is selected from a group consisting of 2-nitobenzyl, benzoin esters, N-acyl-7-nitindolines, meta-phenol, phenacyls and derivatives thereof. In some embodiments the photo-removable protecting group is a 4,5-dimethoxy-2-nitrobenzyl (DMNB) or a derivative thereof. In some embodiments the photo-removable protecting group is covalently attached to the ligand. In some embodiments two or more different targeting ligands are attached to the surface of the particles. In some embodiments at least one of the targeting ligands is tissue specific. In some embodiments the targeting ligand is cell-type specific. In some embodiments the cell type is selected from the group consisting of: HUVECs, MSCs, fibroblasts, cardiomyocytes and human embryonic stem cells (hESCs).

It should be appreciated that an effective amount as used herein in the context of a particle is an amount that is sufficient to achieve a desired medical effect in a subject when a composition comprising a plurality of particles is administered to the subject. In some embodiments, a single particle may be effective if the amount in a single particle is sufficient to have the desired effect. However, typically a plurality of particles are administered to a subject, and an effective amount for each particle is the amount that provides a total cumulative dose sufficient to achieve the desired outcome in the subject based on the number of particles that are administered and the frequency of administration as described in more detail herein.

Each of the limitations of the invention can encompass various embodiments of the invention. It is, therefore, anticipated that each of the limitations of the invention involving any one element or combinations of elements can be included in each aspect of the invention. The invention is capable of other embodiments and of being practiced or of being carried out in various ways. Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

These and other aspects of the inventions, as well as various advantages and utilities will be apparent with reference to the Detailed Description. Each aspect of the invention can encompass various embodiments as will be understood.

All documents identified in this application are incorporated in their entirety herein by reference.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing.

FIG. 1 illustrates a non-limiting embodiment of the instant invention. Non-specific (target every cell type) targeters on the surface of nanoparticles are caged to become non-functional. Upon light illumination, the caging group is released, the targeter is activated and the nanoparticle can bind any tissue where light is applied.

FIG. 2 depicts a non-limiting embodiment of the inactive (Panel A) and active peptide (Panel B) comprising the YIGSR (SEQ ID NO: 1) motif. The GGGGYIGSR-NH2 (SEQ ID NO: 2) peptide was caged with 4,5-dimethoxy-2-nitrobenzyl (DMNB). After illumination the caging group is released and the targeter becomes active.

FIG. 3 shows non-limiting embodiments of the retention time in the HPLC column of the non-caged targeter (Panel A), non-illuminated caged targeter (Panel B) and ten second post illumination (Panel C). Retention time in the HPLC column of the non-caged targeter was ˜20 min (FIG. 3A) while that of the non-illuminated caged targeter was ˜30 min (FIG. 3B). Ten seconds post illumination a shift in the retention time had occurred and the targeter had exited the column after ˜20 min (FIG. 3C).

FIG. 4 shows non-limiting embodiments of the release of the caging group from the targeter-conjugated nanoparticles. FIG. 4A follows the disappearance of the ether bond on the targeter as assessed by FTIR, while FIG. 4B follows the free DMNB caging group released to the media post illumination.

FIGS. 5A and 5B are non-limiting embodiments of qualitative assessments of HUVEC targeting in illuminated and non-illuminated cultures. The particles appear in white. FIGS. 5C and 5D are percentage of targeted MSCs and HUVECs, respectively.

FIG. 6 shows a non-limiting image of certain caged nanoparticles. The amine-terminated caged peptides/targeters were conjugated to the surface of carboxyl-terminated polystyrene nanoparticles (328±2 nm) using 1-ethyl-3-(3-dimethylaminopropyl) carbodiimide (EDC) and sulfo-N-hydroxysuccinimide (NHS) activation chemistry.

FIG. 7 demonstrates non-limiting embodiments of targeting of HUVECs. FIG. 7A is a macroscopic view under UV illumination of fluorescent nanoparticles adhering specifically to cells in a small area that had been illuminated at 340 nm for 1 min (arrow). FIG. 7B is a microscopic view of the cells in the illuminated area, while FIG. 7C is a microscopic view of the cells located 1 cm away. Cell cytoplasm was stained with β actin antibody and the nuclei stained by Hoechst. The nanoparticles appear as white specks in FIG. 7B.

### DETAILED DESCRIPTION OF THE INVENTION

Aspects of the invention relate to compositions for delivering agents/substances to a target site by providing a composition that includes a delivery moiety attached to a targeting moiety. The delivery moiety may be a particle that contains the agent/substance being delivered. The targeting moiety may be a targeting ligand that is reversibly inactivated by a mechanism that allows activation of the targeting ligand in situ after the composition is administered to an individual. Reversible inactivation of the targeting ligand may be achieved using one or more light-sensitive, heat-sensitive, pressure-sensitive, and/or pH-sensitive modifications, microwave-sensitive, X-ray sensitive, and/or one or more modifications that are sensitive to one or more other inputs such as one or more other forms of energy input. Aspects of the invention allow for the use of tissue specific and non-specific ligands that can be used to selectively target an area of interest. In some embodiments, the active targeting ligand binds to a target molecule (anti-ligand), for example on a cell surface, thereby attaching and/or concentrating the composition in the vicinity of the anti-ligand (and/or cell or tissue on which the anti-ligand is present).

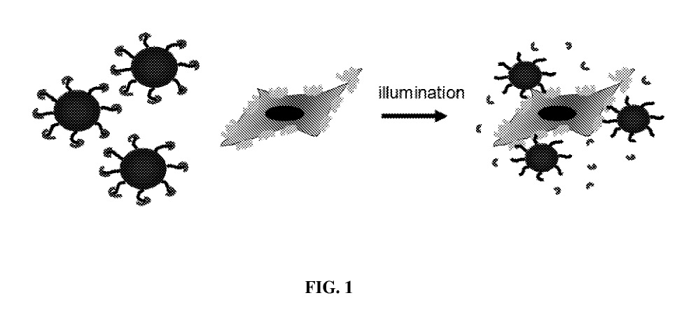

In some embodiments, the present invention is based, at least in part, on a novel particulate system that can target and bind any tissue selectively upon light illumination with a potential of releasing diagnostic and/or therapeutic substances/agents at any desired site (FIG. 1). The first component is the “particle/carrier” that can carry diagnostic and/or therapeutic loadings (e.g., imaging compounds, drugs, growth factors, cytokines etc.). Currently, natural and synthetic polymers and lipids are typically used as drug delivery vectors.

The second component in this system includes “diagnostic and/or therapeutic substances/agents”. The particles can be loaded with a range of substances including drugs, growth factors, chemokines and imaging molecules. The carriers may be used to increase local drug concentration by carrying the drug within and concentrating it and/or control-releasing it when bound to a target.

The third component in this system is the “targeting ligand”. In some embodiments, the targeting ligand is inactivated by caging using a photo-removable protecting group. In some embodiments, the inactive ligand is a caged macromolecule, (e.g., one or more caged peptides, antibodies, aptamers, receptors and/or antigens). The idea behind the caging technique is that a targeting ligand can be temporarily rendered biologically non-functional (or caged) by chemical modification with a photo-removable protecting group. Irradiation can be used to release the protecting group from the ligand surface and restore its ability to attach to a anti-ligand, for example, on a cell of interest. In some embodiments, the anti-ligand is the natural binding partner of the ligand. For example, the anti-ligand may be a surface receptor on a cell and the targeting ligand is the natural ligand (or a portion thereof) of the receptor. Accordingly, the targeting ligand may be a natural binding partner (or a binding fragment thereof) of a cell surface molecule (e.g., protein or other cell surface molecule). However, it should be appreciated that in some embodiments the ligand may be a synthetic molecule (e.g., a synthetic peptide, nucleic acid, or other synthetic molecule) that binds to a cell surface molecule (the anti-ligand). It should be appreciated that the targeted anti-ligand may be a naturally occurring molecule. In some embodiments, the targeted anti-ligand may be cell or tissue specific (e.g., preferentially or uniquely present on specific cells or tissue). In certain embodiments, an anti-ligand may be naturally present on two or more cell or tissue types (e.g., not cell or tissue specific). In some embodiments, an anti-ligand may be specific for a particular condition (e.g., a disease state, for example a variant molecule associated with a disease such as cancer). In some embodiments, the anti-ligand may be a receptor, channel protein, glycoprotein, proteoglycan, adhesion molecule or any other cell surface molecule. In some embodiments, the anti-ligand may be a gap junction protein such as connecin 43, a channel such as an ion channel and/or an ATP channel, an adhesive such as CD31 (VECAM), N-cadherin, VE cadherin, and/or E cadherin, a glycoprotein such as CD44 and/or CD133, a receptor such as VEGFR2 and/or angiotensin and a proteoglycan such as heparan sulfate and/or aggrecan.

In some embodiments, the invention relates to a composition comprising a plurality of particles that contain an effective amount of a diagnostic and/or therapeutic substance. A targeting ligand inactivated by caging can be attached to the surface of the particles. The inactivated ligand can be activated by the removal of the caging group by irradiation of the composition (for example, in situ after administration to a subject, e.g., a human subject). In some embodiments, other forms of energy may be used to activate the ligand that has been caged using other techniques. In some embodiments, the particles do not contain any diagnostic and/or therapeutic substance. Accordingly, in some embodiments, a particle attached to a ligand may be provided so that it can be loaded with a substance of interest. The ligand may be caged or not caged prior to the particle being loaded.

In some embodiments, the invention relates to a method for targeted delivery of a substance to predefined cells or tissues using a composition as described above. In some embodiment two or more different targeting ligands are attached to the surface of the particles. The targeting ligands may be tissue specific or non-specific. In some embodiments, the targeting ligands may be found only on a specific cell type. In some embodiments, the anti-ligands may be receptors, channel proteins, glycoproteins, proteoglycans, adhesion molecules or any other cell surface molecules.

Aspects of the invention may be useful for targeted delivery of drugs, and for targeting cells which do not have any unique biomarkers. The technology allows spatial and temporal specificity to be conferred on a non-specific targeting ligand. Methods of the invention provide for rapid and localized release of molecules of interest to any tissue in the body. Compounds and methods of the invention allow delivery of therapeutic compositions to discrete regions of the body by virtue of the ability to activate caged targeting ligands by a focused beam of light (e.g., ultraviolet or infrared) or other energy source. For example, this approach may be used for targeted delivery to the eye, skin, and ear and also can be used for treating other internal organs with the aid of minimally invasive fiber optic technology or other optical (e.g., near infrared) or other activation technology that can penetrate the body of a subject to activate the targeting ligand in a region of interest (e.g., adjacent to a site of disease, for example near a tumor or other cancerous tissue). The approach could also be used to bind injected or implanted devices bearing a molecule of interest. The latter has many potential uses, such as the problem of reloading the drug content of implanted drug delivery systems, treating infected hardware, etc.

Aspects of the invention may also be useful to transfer drugs across the blood brain barrier. The compositions of the invention may be produced using targeting ligands which can bind specific anti-ligands present at the blood brain barrier. In some embodiments, the targeting ligand is transferrin or insulin. In some embodiments, tissue non-specific targeting ligands are used in combination with the tissue-specific ligands.

Accordingly, aspects of the invention may be used to target therapeutic, diagnostic/imaging, and/or other molecules to any target site of interest in a subject. For example, a composition may be selectively activated at a site of diseased tissue anywhere in the body of a subject. In some embodiments, the target may be in or near an organ that is diseased (e.g., cancerous). In some embodiments, the target may be a portion of a tissue or organ. For example, a composition may be activated in or near the liver, pancreas, lung, colon, bladder, cervix, heart, bone, kidney, bone tissue, muscle tissue, or a portion thereof. In some embodiments, vascular tissue in or near an organ or target tissue of interest may be targeted for activation (e.g., using light or other energy source described herein). It should be appreciated that aspects of the invention may be used to treat or diagnose (or assist in the treatment or diagnosis) of a multicellular organism, for example, a vertebrate, a mammal (e.g., a human, an agricultural or domestic mammal) or other animal. It should be appreciated that compositions of the invention may be administered in any suitable way. In some embodiments, a composition may be injected, administered orally, or otherwise administered. In some embodiments, a composition may be administered intravenously, intraperitoneally, or otherwise. Accordingly, in some embodiments, a composition may be provided systemically. In some embodiments, a composition may be provided locally. It should be appreciated that a composition may be activated locally, at one or more locations, or more generally in a subject (e.g., a patient in need of diagnosis and/or treatment).

It should be appreciated that one or more diagnostic and/or therapeutic agents may be administered to a subject in an effective amount. An effective amount of an agent is a dose sufficient to provide a medically desirable result and can be determined by one of skill in the art using routine methods. In some embodiments, an effective amount is an amount which results in any improvement in the condition being treated. In some embodiments, an effective amount may depend on the type and extent of disease or condition being treated and/or use of one or more additional therapeutic agents. However, one of skill in the art can determine appropriate doses and ranges of therapeutic agents to use, for example based on in vitro and/or in vivo testing and/or other knowledge of compound dosages. Similarly, effective amounts of a diagnostic agent can be determined based on the desired diagnostic application. Accordingly, since agents described herein are being administered in particles, an effective amount for each particle is an amount sufficient to contribute to a total effective amount of agent when taking into account the number of particles that are administered to a subject and the frequency of administration.

When administered to a subject, effective amounts of a therapeutic agent will depend, of course, on the particular disease being treated; the severity of the disease; individual patient parameters including age, physical condition, size and weight, concurrent treatment, frequency of treatment, and the mode of administration. These factors are well known to those of ordinary skill in the art and can be addressed with no more than routine experimentation. In some embodiments, a maximum dose is used, that is, the highest safe dose according to sound medical judgment. Similarly, effective amounts of a diagnostic agent can depend on one or more parameters, including the age, physical condition, size, weight, and other medical conditions of a subject.

In some embodiments, an effective amount of a therapeutic or diagnostic agent typically will vary from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, from about 10.0 mg/kg to about 150 mg/kg in one or more dose administrations, for one or several days (depending of course of the mode of administration and the factors discussed above). Accordingly, the effective amount of an agent to be loaded in a particle described herein (e.g., a targeted particle) will depend on the number of particles and frequency of particle administration to a subject. It should be appreciated that one of skill in the art can determine appropriate therapeutic and/or diagnostic regimens based on the amount of agent that is loaded per particle, the number of particles that are administered to a subject in each dose, and the frequency of administration. In some embodiments, each of these parameters may be varied to deliver a desired (e.g., effective) amount of agent(s) to a subject (e.g., a human subject). In some embodiments, the number of particles administered in a single dose may be in the range of 100 to 10²⁰.

Actual dosage levels of a diagnostic or therapeutic agent can be varied (e.g., by varying the amount per particle, the frequency of administration, the number of particles that are administered, or a combination thereof) to obtain an amount that is effective to achieve the desired therapeutic response for a particular patient, compositions, and mode of administration. The selected dosage level depends upon the activity of the particular agent, the route of administration, the tissue being treated, and prior medical history of the patient being treated. However, it is within the skill of the art to start doses of the agent (e.g., doses achieved using a plurality of particles) at levels lower than required to achieve the desired therapeutic effort and to gradually increase the dosage until the desired effect is achieved.

### A. Particles/Carriers

In certain embodiments of the invention the “particles” of the invention comprise a biocompatible polymer, which preferably is biodegradable. Suitable polymers include, but are not limited to, poly(lactic-co-glycolic acid), polyanhydrides, ethylene vinyl acetate, polyglycolic acid, chitosan, polyorthoesters, polyethers, polylactic acid, and poly (beta amino esters). Peptides, proteins such as collagen, and dendrimers (e.g., PAMAM dendrimers) can also be used. In certain embodiments of the invention a poly (beta amino ester) compound, or a salt or derivative thereof, is used as a carrier. The carrier can be used in the form of microparticles, nanoparticles, solid drug delivery articles, and/or as a soluble nanometer scale complex with a nucleic acid.

In certain embodiments of the invention the particles may be drug delivery devices comprising a solid material such as polymeric matrix impregnated with, or encapsulating, a therapeutic agent. The device is implanted into the body at the location of the target tissue or in the vicinity thereof, or in a location distant from the target tissue. The therapeutic agent is released from the polymeric matrix upon light irradiation. The therapeutic agent can be released by diffusion, degradation of the polymeric matrix or cellular uptake.

A polymeric matrix comprising the particle of the invention may assume a number of different shapes. For example, microparticles of various sizes (which may also be referred to as beads, microbeads, microspheres, nanoparticles, nanobeads, nanospheres, etc.) can be used. Polymeric microparticles and their use for drug delivery are well known in the art. Such particles are typically approximately spherical in shape but may have irregular shapes. Generally, a microparticle will have a diameter of 500 microns or less, e.g., between 50 and 500 microns, between 20 and 50 microns, between 1 and 20 microns, between 1 and 10 microns, and a nanoparticle will have a diameter of less than 1 micron. If the shape of the particle is irregular, then the volume will typically correspond to that of microspheres or nanospheres. The polymeric matrix can be formed into various nonparticulate shapes such as wafers, disks, rods, etc., which may have a range of different sizes and volumes. Methods for incorporating therapeutically active agents into polymeric matrices are known in the art.

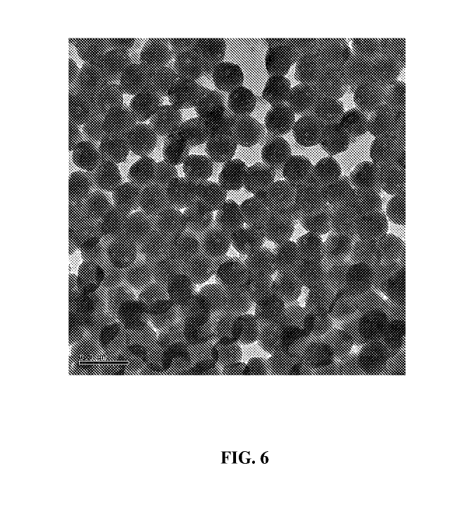

Solid nanoparticles or microparticles can be made using any method known in the art including, but not limited to, spray drying, phase separation, single and double emulsion solvent evaporation, solvent extraction, and simple and complex coacervation. Certain methods include spray drying and the double emulsion process. Solid agent-containing polymeric compositions can also be made using granulation, extrusion, and/or spheronization. The nanoparticles used in the present invention are well known in the art and include those described in detail in Mallidi, S. et al. Nano Letters 2009, 9, (8), 2825-31; Bagalkot, V. et al. Nano Lett 2007, 7, (10), 3065-70; and Farokhzad, O. C. et al. Proc Natl Acad Sci USA 2006, 103, (16), 6315-20. In some embodiments, the nanoparticles are liposomes. In some embodiments, the nanoparticles are carboxyl-terminated polystyrene nanoparticles. In some embodiments, the carboxyl-terminated polystyrene nanoparticles have a diameter of 328±2 nm (FIG. 6).

The conditions used in preparing the microparticles may be altered to yield particles of a desired size or property (e.g., hydrophobicity, hydrophilicity, external morphology, “stickiness”, shape, etc.). The method of preparing the particle and the conditions (e.g., solvent, temperature, concentration, air flow rate, etc.) used may also depend on the agent being encapsulated and/or the composition of the polymer matrix. If the particles prepared by any of the above methods have a size range outside of the desired range, the particles can be sized, for example, using a sieve or other size separation technique. Methods developed for making microparticles for delivery of encapsulated agents are described in the literature.

Solid polymer-agent compositions (e.g., disks, wafers, tubes, sheets, rods, etc.) can be prepared using any of a variety of methods that are well known in the art. For example, in the case of polymers that have a melting point below the temperature at which the composition is to be delivered and/or at which the polymer degrades or becomes undesirably reactive, a polymer can be melted, mixed with the agent to be delivered, and then solidified by cooling. A solid article can be prepared by solvent casting, in which the polymer is dissolved in a solvent, and the agent is dissolved or dispersed in the polymer solution. Following evaporation of the solvent, the substance is left in the polymeric matrix. This approach generally requires that the polymer is soluble in organic solvent(s) and that the agent is soluble or dispersible in the solvent. In still other methods, a powder of the polymer is mixed with the agent and then compressed to form an implant.

Many of the useful polymers contain both chargeable amino groups, to allow for ionic interaction with the negatively charged DNA phosphate, and a degradable region, such as a hydrolyzable ester linkage. Examples of these include poly(alpha-(4-aminobutyl)-L-glycolic acid), network poly(amino ester), and poly (beta-amino esters). These complexation agents can protect DNA against degradation, e.g., by nucleases, serum components, etc., and create a less negative surface charge, which may facilitate passage through hydrophobic membranes (e.g., cytoplasmic, lysosomal, endosomal, nuclear) of the cell. Certain complexation agents facilitate intracellular trafficking events such as endosomal escape, cytoplasmic transport, and nuclear entry, and can dissociate from the nucleic acid. It has been proposed that such agents may act as a “proton sponge” within the endosome.

### B. Diagnostic/Therapeutic Agents

A wide variety of “diagnostic and/or therapeutic agents” may be incorporated into the particles. By “therapeutic”, as used herein, it is meant an agent having a beneficial effect on the patient. As used herein, the term therapeutic is synonymous with the term drug. Suitable therapeutics include, but are not limited to: antineoplastic agents, hormones, cytokines, cytotoxins, anti-microbial agents (anti-fungals, anti-virals, antiprotozoans), antibiotics, vitamins, antituberculars, antirheumatics, anti-allergic agents, circulatory drugs, antianginals, anticoagulants, narcotics, cardiac glycosides, neuromuscular blockers, sedatives (hypnotics), and local and general anesthetics.

Anti-neoplastic agents include, but are not limited to, platinum compounds (e.g., spiroplatin, cisplatin, and carboplatin), methotrexate, adriamycin, mitomycin, ansamitocin, bleomycin, cytosine arabinoside, arabinosyl adenine, mercaptopolylysine, vincristine, busulfan, chlorambucil, melphalan (e.g., PAM, L-PAM or phenylalanine mustard), mercaptopurine, mitotane, procarbazine hydrochloride dactinomycin (actinomycin D), daunorubicin hydrochloride, doxorubicin hydrochloride, taxol, mitomycin, plicamycin (mithramycin), aminoglutethimide, estramustine phosphate sodium, flutamide, leuprolide acetate, megestrol acetate, tamoxifen citrate, testolactone, trilostane, amsacrine (m-AMSA), asparaginase (L-asparaginase) Erwina asparaginase, etoposide (VP-16), interferon ct-2a, interferon a-2b, teniposide (VM-26), vinblastine sulfate (VLB), vincristine sulfate, bleomycin, bleomycin sulfate, methotrexate, adriamycin, and arabinosyl.

Examples of hormones include, but are not limited to, growth hormone, melanocyte stimulating hormone, estradiol, beclomethasone dipropionate, betamethasone, betamethasone acetate and betamethasone sodium phosphate, vetamethasone disodium phosphate, vetamethasone sodium phosphate, cortisone acetate, dexamethasone, dexamethasone acetate, dexamethasone sodium phosphate, flunisolide, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, paramethasone acetate, prednisolone, prednisolone acetate, prednisolone sodium phosphate, prednisolone tebutate, prednisone, triamcinolone, triamcinolone acetonide, triamcinolone diacetate, triamcinolone hexacetonide and fludrocortisone acetate.

Potentially useful ctokines include, but are not limited to, lymphokines, interleukins, interferons, and chemokines.

Examples of cytotoxins contemplated include, but are not limited to, cholera toxin, ricin, LT-toxin, C3 toxin, Shiga toxin, pertussis toxin, tetanus toxin, saporin, modeccin, gelanin and tumor necrosis factor.

Non-limiting examples of antimicrobials include antivirals such as acyclovir, amantadine azidothymidine (AZT or Zidovudine), ribavirin and vidarabine monohydrate (adenine arabinoside, ara-A); anti-fungal agents such as ketoconazole, nystatin, griseofulvin, flucytosine (5-fc), miconazole, amphotericin B, ricin, and pMactam antibiotics (e.g., sulfazecin); antiprotozoans such as chloroquine, hydroxychloroquine, metronidazole, quinine and meglumine antimonate; and biological response modifiers such as muramyldipeptide, muramyltripeptide, microbial cell wall components, lymphokines (e.g., bacterial endotoxin such as lipopolysaccharide, macrophage activation factor), sub-units of bacteria (such as Mycobacteria, Corynebacteria), the synthetic dipeptide N-acetyl-muramyl-L-alanyl-D-isoglutamine.

Antibiotics include, but are not limited to, dapsone, chloramphenicol, neomycin, cefaclor, cefadroxil, cephalexin, cephradine erythromycin, clindamycin, lincomycin, amoxicillin, ampicillin, bacampicillin, carbenicillin, dicloxacillin, cyclacillin, picloxacillin, hetacillin, methicillin, nafcillin, oxacillin, penicillin G, penicillin V, ticarcillin rifampin and tetracycline.

Examples of anti-inflammatories include, but are not limited to diflunisal, ibuprofen, indomethacin, meclofenamate, mefenamic acid, naproxen, oxyphenbutazone, phenylbutazone, piroxicam, sulindac, tolmetin, aspirin and salicylates.

Examples of vitamins include but are not limited to cyanocobalamin neinoic acid, retinoids and derivatives such as retinol palmitate, and α-tocopherol.

Examples of antituberculars include but are not limited to para-aminosalicylic acid, isoniazid, capreomycin sulfate cycloserine, ethambutol hydrochloride ethionamide, pyrazinamide, rifampin, and streptomycin sulfate.

Examples of antirheumatics include but are not limited to penicillamine.

Examples of anti-allergic agents include but are not limited to amelexanox; anti-coagulation agents such as phenprocoumon and heparin.

Examples of circulatory drugs include but are not limited to propranolol; metabolic potentiators such as glutathione.

Examples of antianginals include but are not limited to diltiazem, nifedipine, verapamil, erythritol tetranitrate, isosorbide dinitrate, nitroglycerin (glyceryl trinitrate) and pentaerythritol tetranitrate.

Examples of anticoagulants include but are not limited to phenprocoumon, heparin.

Examples of narcotics include but are not limited to paregoric; opiates such as codeine, heroin, methadone, morphine and opium.

Examples of cardiac glycosides include but are not limited to deslanoside, digitoxin, digoxin, digitalin and digitalis.

Examples of neuromuscular blockers include but are not limited to atracurium mesylate, gallamine triethiodide, hexafluorenium bromide, metocurine iodide, pancuronium bromide, succinylcholine chloride (suxamethonium chloride), tubocurarine chloride and vecuronium bromide.

Examples of sedatives (hypnotics) include but are not limited to amobarbital, amobarbital sodium, aprobarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, pentobarbital sodium, phenobarbital sodium, secobarbital sodium, talbutal, temazepam and triazolam.

Examples of local anesthetics include but are not limited to bupivacaine hydrochloride, chloroprocaine hydrochloride, etidocaine hydrochloride, lidocaine hydrochloride, mepivacaine hydrochloride, procaine hydrochloride and tetracaine hydrochloride; general anesthetics include but are not limited to droperidol, etomidate, fentanyl citrate with droperidol, ketamine hydrochloride, methohexital sodium and thiopental sodium; and radioactive particles or ions such as strontium, iodide rhenium and yttrium.

Other therapeutics include genetic material such as nucleic acids, RNA, and DNA, of either natural or synthetic origin, including recombinant RNA and DNA, antisense RNA and DNA and siRNA or other small RNA. Types of genetic material that may be used include, for example, genes carried on expression vectors such as plasmids, phagemids, cosmids, yeast artificial chromosomes (YACs), and defective or “helper” viruses, antigene nucleic acids, both single and double stranded RNA and DNA and analogs thereof, such as phosphorothioate and phosphorodithioate oligodeoxynucleotides. Additionally, the genetic material may be combined, for example, with proteins or other polymers.

If desired, more than one therapeutic may be applied using the microspheres. For example, a single microsphere may contain more than one therapeutic or microspheres containing different therapeutics may be co-administered.

As used herein, “diagnostic agent” comprises any agent that can be used in the diagnosis of a disease in an individual. Non-limiting examples include imaging agents such as radioisotopes, dyes, pigments and fluorescent molecules (such as luciferase, and fluorescein) and heavy metals (such as gadolinium).

Accordingly, it should be appreciated that a diagnostic or therapeutic agent may be a peptide, protein, nucleic acid (DNA or RNA), small molecule, or any combination thereof.

### C. Targeting Ligands

As used herein, the “targeting ligand” comprises any type of molecule for which there exists another molecule (e.g., an “anti-ligand”) that binds to the ligand, owing to a favorable (i.e., negative) change in free energy upon contact between the ligand and anti-ligand. The binding between the ligand and anti-ligand can be specific with binding affinities in the micromolar to picomolar range. Ligand/anti-ligand pairs may be a antigen/antibody, enzyme/substrate, DNA/DNA, DNA/RNA, RNA/RNA, nucleic acid mismatches, complementary nucleic acids and nucleic acid/proteins. It will be appreciated that any molecule can act either as a ligand or an anti-ligand. In some embodiments, the ligand comprises a peptide, an antibody, an aptamer, a receptor or an antigen. The targeting ligand may be inactivated by caging using a photo-removable protecting group, heat-sensitive group, pressure-sensitive group, microwave-sensitive, a pH sensitive group or any other group that can be removed upon exposure to a suitable energy source. The ligand may be tissue specific or non-specific. In some embodiments, the ligand is tissue non-specific.

In some embodiments, the targeting ligand may be inactivated by caging using a photo-removable protecting group. In general, caging using any suitable technique (e.g. using a photoremovable group or any other suitable group) inhibits or conceals (e.g., by disrupting bonds that normally stabilize an interaction with a target molecule, by modifying the hydrophobicity or ionic character of a particular side chain of the ligand, or by steric hindrance) an important property necessary for biological activity, e.g., an active site or a folding pattern, any combination thereof. In some embodiments, the presence of the caging group on the targeting ligand will change its conformation and thus will prevent recognition of the ligand by its anti-ligand found on cell surface. Removal of the caging group activates the ligand. The targeting ligand is covalently attached to the surface of a particle.

In some embodiments, the ligand comprises a peptide, an antibody, an aptamer, a receptor or an antigen. In some embodiments, the ligand is a peptide comprising an amino acid sequence containing a motif known to be vital for integrin-receptor mediated cell attachment. As such, the peptide can be temporarily rendered biologically non-functional relative to the corresponding peptide by caging using a photo-removable protecting group (or other removable group). The “inactivated peptide” is an above-indicated “peptide” which is rendered biologically inactive by covalent modification (e.g., caging) by the attachment of a photo-removable protecting group (or other removable group). The “inactivated peptide/particle adduct” comprises an “inactivated peptide” which is covalently attached to the surface of a particle comprising a substance of interest.

It should be appreciated that in some embodiments the ligand may be any suitable molecule (e.g., a peptide) that binds to a cell surface molecule (anti-ligand). The cell surface molecule may be a protein receptor or other cell surface protein that is capable of binding to a specific ligand (either a natural or synthetic ligand).

In some embodiments, the targeting ligand may be specific for an anti-ligand that is present on an endothelial cell (e.g., a surface antigen on an endothelial cell). Accordingly, upon activation of the targeting ligand, an attached particle may bind to an endothelial cell. In some embodiments, this allows particles in a blood vessel to be activated to bind to endothelial cells in the blood vessel wall. In some embodiments, particles that bind to an area of a blood vessel wall may cross the endothelial layer and deliver an agent or other substance to a tissue or organ adjacent to the area of the blood vessel. It should be appreciated that the anti-ligand on the endothelial cell may be an endothelial-specific molecule. However, in some embodiments, it may be a molecule that is present on endothelial cells in addition to other cells. According to the invention, binding to a target region on a blood vessel wall may be accomplished by activating the ligand in the vicinity of the target region. It should be appreciated that in some embodiments the targeting ligand of a composition of the invention may be activated in a blood vessel (e.g., by light) upstream of the target region (for example, if the kinetics of ligand activation and binding would result in binding within the target region even though activation occurred upstream of the target region, because of blood flow taking the activated composition from the activation region to the target region).

In some embodiments, the anti-ligand may be a receptor, channel protein, glycoprotein, proteoglycan, adhesion molecule or any other cell surface molecule. In some embodiments, the anti-ligand may be a gap junction protein such as connecin 43, a channel such as an ion channel and/or an ATP channel, an adhesive such as CD31 (VECAM), N-cadherin, VE cadherin, and/or E cadherin, a glycoprotein such as CD44 and/or CD133, a receptor such as VEGFR2 and/or angiotensin and a proteoglycan such as heparan sulfate and/or aggrecan.

In some embodiments the inactivated peptide/particle adduct is prepared from a peptide which is first caged with a photo-removable protecting group, followed by covalent attachment of the caged peptide to the particle. In other embodiments the inactivated peptide/particle adduct is prepared from a covalent attachment of the particle to the peptide during the first step, followed by the caging of the peptide portion of the peptide/particle adduct with a photo-removable protecting group. In further embodiments, the inactivated peptide/particle adduct is prepared from a “one-pot” single-step reaction of the peptide, the nanoparticle, and the photo-removable protecting group.

In some embodiments, the peptide comprises a RGD motif of fibronectin. In other embodiments, the peptide comprises a YIGSR (SEQ ID NO: 1) motif of laminin. In some embodiments, the peptide comprises synthetic YIGSR-containing peptides such as CDPGYIGSR (SEQ ID NO: 3) and/or YIGSR-NH₂ (SEQ ID NO: 1). The photo-removable protecting groups used in the present invention are well known in the art (Pillai, in Organic Photochemistry, Vol. 9, A Padwa, ed., Marcel Dekker, Inc., New York, 1987, pp. 225-323). Examples of suitable photo-removable protecting groups include, but are not limited to, 2-nitobenzyl, benzoin esters, N-acyl-7-nitindolines, meta-phenols, phenacyls and derivatives thereof. In some embodiments, the photo-removable protecting group is a 2-nitrobenzyl derivative, such as a 4,5-dimethoxy-2-nitrobenzyl (DMNB) derivative.

In some embodiments, a hydroxyl (—OH) substituent of the peptide reacts with the photo-removable protecting group. In other embodiments an amino (—NH₂, or —NH—) substituent of the peptide reacts with the photo-removable protecting group. In some embodiments, a thiol (—SH) substituent of the peptide reacts with the photo-removable protecting group. In other embodiments a carboxylic acid (—CO₂H) substituent or a derivative thereof, such as an ester (—CO₂-Aliphatic) substituent, of the peptide reacts with the photo-removable protecting group.

In some embodiments, the hydroxyl (—OH) substituent of the peptide which reacts with the photo-removable protecting group is derived from the side chain of serine, threonine, tyrosine, or hydroxyproline. In other embodiments, the amino (—NH₂, or —NH—) substituent of the peptide which reacts with the photo-removable protecting group is derived from the side chain of tryptophan, histidine, arginine, lysine, or ornithine. In some embodiments, the thiol (—SH) substituent of the peptide which reacts with the photo-removable protecting group is derived from the side chain of cystine. In other embodiments, the carboxylic acid (—CO₂H) substituent or a derivative thereof, such as an ester (—CO₂-Aliphatic) substituent, of the peptide which reacts with the photo-removable protecting group is derived from the side chain of aspartic acid or glutamic acid.

In some embodiments an amino (—NH₂, or —NH—) substituent of the peptide or caged peptide reacts with the nanoparticle. In other embodiments a carboxylic acid (—CO₂H) substituent or a protected carboxylic acid derivative (—CO₂-Aliphatic) substituent of the peptide or caged peptide reacts with the nanoparticle.

In some embodiments an amino (—NH₂, or —NH—) substituent of the nanoparticle reacts with the peptide or caged peptide. In other embodiments a carboxylic acid (—CO₂H) substituent or a protected carboxylic acid derivative (—CO₂-Aliphatic) substituent of the nanoparticle reacts with the peptide or caged peptide.

In some embodiments, the peptides, photo-removable protecting groups, and nanoparticles of the invention are covalently attached according to synthetic methods which are well known in the art and include those described in detail in Protecting Groups in Organic Synthesis, T. W. Greene and P. G. M. Wuts, 3^{rd} edition, John Wiley & Sons, 1999; and Chemistry of Peptide Synthesis, N. Leo Benoiton, CRC Press, 2005; Smith and March March's Advanced Organic Chemistry, 5^{th} Edition, John Wiley & Sons, Inc., New York, 2001; and Larock, Comprehensive Organic Transformations, VCH Publishers, Inc., New York, 1989; the entirety of which are incorporated herein by reference.

In some embodiments, side chain heteroatoms (O, N, or S) of the peptides are covalently attached to the benzyl positions of photo-removable protecting groups. In some embodiments, sidechain heteroatoms (O, N, or S) of the peptides react with a nitrobenzyl halide derivative, such as 4,5-dimethoxy-2-nitrobenzyl chloride or 4,5-dimethoxy-2-nitrobenzyl bromide.

In some embodiments the peptides or the caged peptides of the invention are covalently attached to the nanoparticles via amide bonds. In some embodiments, the amide bonds are formed from an amine group of the peptides or the caged peptides of the invention and the carboxylic acid substituents of the nanoparticles. In some embodiments, the amide bond is formed from sulfo-N-hydroxysuccinimide (NHS) and/or 1-ethyl-3-(3-dimethylaminopropyl) carbodiimide (EDC).

It should be appreciated that the same or equivalent chemical modifications may be made on other types of ligands (e.g., non-peptide ligands) to cage them. It should also be appreciated that the same or similar chemical modifications may be used to attach any suitable removable group in addition to or instead of a photo-removable group.

Activation of the inactivated targeting ligand can be accomplished upon exposure to light, heat, pressure, microwaves, a change in pH, a change in the level of one or more metabolites, and/or other sources of energy. In some embodiments, the protecting group on the ligand is removed upon exposure to any suitable conventional light source. Examples of such light source include, without limitation, lasers, (e.g., excimer lasers) emitting energy in the ultraviolet portion of the spectrum or lasers (e.g., diode, Ti:sapphire lasers, holmium lasers (and other rare earth metal lasers), neodynium (Nd) YAG, Nd YAG lasers) emitting radiation in the infrared portion of the spectrum, and which produce brief, high flux density emissions. If desired, pulsed irradiation, which is useful in generating two-photon excitation, can be generated by standard optical modulation techniques known in the art, such as by employing mode-locked lasers (using, for example, electro or acousto-optic devices). Lasers that operate in a pulsed mode in the infrared, visible, and nearinfrared spectrum include Nd:YAG, Nd:YLF, CO2, excimer, dye, Ti:sapphire, diode, holmium (and other rare-earth materials), and metal-vapor lasers. The pulse widths of these light sources are adjustable, and can vary from several tens of femtoseconds to several hundred microseconds.

In general, lasers are preferable sources of irradiation because they provide well defined spatially coherent wave-lengths of irradiation particularly suited for uncaging of photosensitive caging groups in defined regions. Furthermore, such light sources can be delivered by optical fibers and used to irradiate a specific region in a controllable manner. Fiber optic delivery systems are particularly maneuverable, and can be used to irradiate a region of the body, e.g., a tissue, thereby generating irradiation in hard to reach places. These types of delivery systems, when optically coupled to lasers, are useful as they can be integrated into catheters and related flexible devices, and used to irradiate virtually any organs or region in the body (e.g., human body). In addition, the wavelength of the optical source can be easily tailored to generate the appropriate absorption in a particular cell or tissue type; this allows a number of different cells or tissues to be effectively treated using the compounds and methods of the invention. In some embodiments, the wavelength of light used is between 350-400 nm. In some embodiments, near infra irradiation is used.

Photolysis of photosensitive caged peptides affords a means of controlling the release, both spatially and temporally, of biologically active peptides or other molecules. In particular, photolysis of caged molecules (e.g., peptides) of the invention can be localized with precision to discrete regions of a cell or tissue of the body by virtue of the ability to activate the caged product using a focused beam of irradiation, e.g., ultraviolet or infrared irradiation. In the latter case, it is useful to employ high flux densities for facilitating two photon excitation (Denk et al., Science 248: 73-76, 1990), which is particularly advantageous for photodynamic therapies employing the caged molecules (e.g., peptides) of the invention, because the tissues of the body are virtually opaque to ultraviolet radiation but transparent to infrared radiation. This method allows beams of light to be focused within the body, thereby controlling reactions at specific sites. A further advantage of two photon excitation methodologies is that the probability of photoactivation of a compound is a function of the square of the distribution of illumination intensity giving rise to a highly defined region for activation. Moreover, the ability of two photon excitation to utilize light in the infrared portion of the spectrum is advantageous since it provides the opportunity to use a wide range of wavelengths that are transmitted within the body.

The present invention is further illustrated by the following Example, which in no way should be construed as further limiting. The entire contents of all of the references (including literature references, issued patents, published patent applications, and co-pending patent applications) cited throughout this application are hereby expressly incorporated by reference.

### EXAMPLE

### Materials and Methods

Assessment of Targeter Un-Caging Potential.

HPLC assays were performed on an HP 1100 HPLC system. Samples were injected in 50-μl volumes onto a C18 column. The column was eluted with an aqueous solution at 1 ml/min. The peptides were detected by a UV detector with absorbance wavelength set at 230 nm.

Synthesis of the Caged Particles.

One milligram of fluorescent polystyrene carboxylated nanoparticle suspension (328±2 nm, Merck Chimie S.A.S, Pithiviers, FR) were incubated with 100 mg of 1-(3-dimethylaminopropyl)-3-ethylcarbodimide hydrochloride (EDC, Sigma) and 200 mg of sulfo-N-hydroxysuccinimide (NHS, Sigma) for 2.5 hours at room temperature with gentle stiffing. The resulting NHS-activated particles were covalently linked to 5 mg NH₂-GGGGY(DMNB)IGSR-NH₂ (SEQ ID NO: 2) peptide (Purity>96% according to HPLC, custom synthesized by Peptech Corp. Burlington, Mass.) over-night at room temperature with gentle stirring. NH₂-GGGGYIGSR-NH₂ and the scrambled peptide NH₂-GGGGFHPDYRVI-NH₂ (SEQ ID NO: 4) (GenScript Corp. Piscataway, N.J.) served as control targeters.

Fourier Transform IR.

Nanoparticle solutions (200 μg/mL) were illuminated for 0, 1 and 5 sec (365 nm: Entela, Upland, Calif.) in a 6-well plate. The solution was collected, centrifuged and the media was discarded. The nanoparticles were then lyophilized for 24 h and FTIR spectroscopy (Bruker Alpha-E, Billerica Mass.) was used to collect their spectra. Non caged particles served as control.

Cell Isolation.

Mesenchymal stem cells (MSCs) were isolated as described in Barbash et al., Circulation 2003, 108, (7), 863-8. Briefly, under sterile conditions, the femur and tibia of 2-3-month-old Sprague-Dawley rats (Charles River, Wilmington, Mass.) were excised. Bone marrow plugs were extracted from the bones by flushing the bone marrow cavity with culture medium. After a homogenous cell suspension was achieved, the cells were centrifuged (600 g, 5 min), resuspended in DMEM and plated (50×10⁶ cells per 75-cm² culture flask). Three days later the media was replaced and adherent cells were considered MSCs. Second-passage BM-MSCs were used in all experiments. Cardiac cells were isolated as described before (Dvir et al., Tissue Eng 2006, 12, (10), 2843-52). Briefly, isolated ventricles were placed in cold Dulbecco's modified Eagle's medium (DMEM)-based buffer (calcium chloride dihydrate, 1.8 mM; potassium chloride, 5.36 mM; magnesium sulfate heptahydrate, 0.81 mM; sodium chloride, 0.1M; sodium bicarbonate, 0.44 mM; sodiumdihydrogen phosphate, 0.9 mM; pH 7.4), cut to approximately 1 mm³ pieces and incubated (37° C., 30 min) repeatedly (6-7 times) in buffer with collagenase type II (95 U/mL; Worthington, Lakewood, N.J.) and pancreatin (0.6 mg/mL; Sigma). After each digestion round, the mixture was centrifuged (600 g, 5 min, 25° C.), and the cell pellet was re-suspended in cold M-199 medium.

Cell Culture and Cellular Binding.

The HUVECs (Lonza Walkersville, Inc. Walkersville, Md.), and MSCs were grown in 8-chamber slides in EGM-2 and DMEM respectively, DMEM was supplemented with 100 units/mL aqueous penicillin, 100 g/mL streptomycin, and 10% fetal bovine serum. The cells were grown at concentrations to allow ˜90% confluence. On the day of experiments, cells were washed with pre-warmed PBS and incubated with pre-warmed media with addition of a 20 μg/mL of caged nanoparticles. The cultured were illuminated for 10 sec, incubated for 30 minutes at 37° C., and washed with PBS three times, stained with β actin antibody (Sigma) and visualized using a fluorescent microscopy. The number of targeted cells was quantified by fluorescent microscopy at 20× magnification and divided by total cell number. For qualitative assessment of caged-nanoparticle targeting and spatial experiments media containing 200 μg/mL caged nanoparticles was added to the cell cultures and the culture dishes/flasks were illuminated (UV lamp or inverted Zeiss microscope, Axiovert 200M for qualitatively targeting assessment and spatial targeting, respectively) or not for 1 min before being carefully washed, visualized by UV light transilluminator (TFX-35M, Life Technologies, Paisley, UK) and the images were photo-documented (Kodak digital science electrophoresis documentation and analysis system 120).

Immunofluorescence Staining.

Immunofluorescence stainings were performed as described before. Briefly the samples were fixed and permeabilized in cold methanol, blocked for 1 h at room temperature in DMEM-based buffer containing 5% FBS. After three buffer washes, the samples were incubated for 1 h with anti-13 actin (FITC-conjugated, Sigma) or β1 integrin (R&D Systems) antibodies (1:500, and 1:50, respectively). After incubation, the samples stained with antibody against β1 integrin were washed and incubated for additional 1 h with goat anti-mouse Alexa 488-conjugated antibodies (1:150). For nuclear detection, the cells were incubated for 3 min with Hoechst 33258(Sigma) and washed. Imaging was performed with an inverted Zeiss fluorescence microscope model Axiovert 200M and analysis was performed using AxioVision 4.5.

### Results

Synthetic YIGSR-containing peptides such as CDPGYIGSR (SEQ ID NO: 3) and YIGSR-NH₂ (SEQ ID NO: 1) have been previously shown to promote cell adhesion and migration. Furthermore, the adhesion of cells to laminin has been shown to occur through binding to integrin β1 on the cell membrane. The proposed target (β1 integrin) was found to be present on several cell types including HUVECs, MSCs, fibroblasts, cardiomyocytes and human embryonic stem cells (hESCs). These cells represent the broader range of target cells the nanoparticulate system is feasible for.

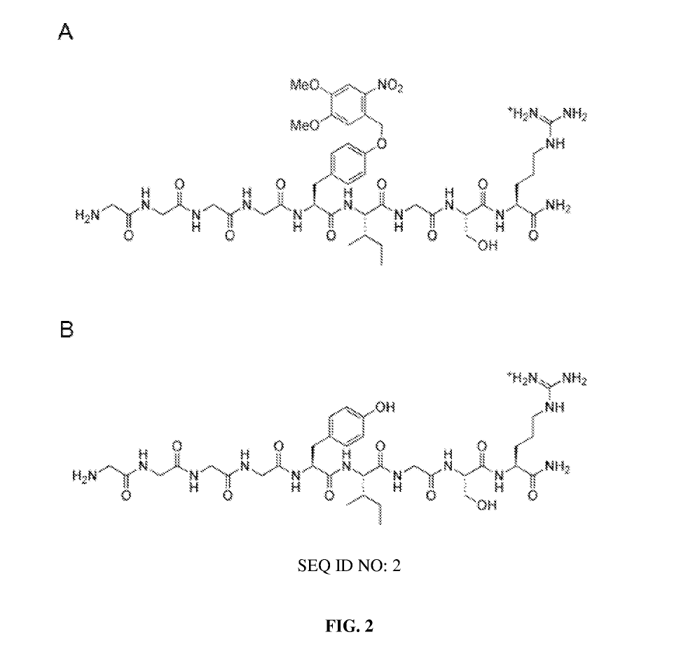

Previously it was shown that mutation or deletion of tyrosine in the YIGSR peptide (SEQ ID NO: 1) resulted in a significant loss of the peptide activity. Therefore, in this research, this amino acid on the YIGSR peptide was caged with 4,5-dimethoxy-2-nitrobenzyl (DMNB) (FIG. 2) and thus inactivated temporarily until it was illuminated. DMNB, the caging group used in this study was chosen since it is well documented in the literature and was shown previously to be released at a msec rate from biological substrate. This may not be the optimal chromophore in terms of un-caging properties and in this study it only served to prove an embodiment of the invention. Important examples of widely used caged compounds, their design features, synthesis and use was previously described by Ellis-Davis.

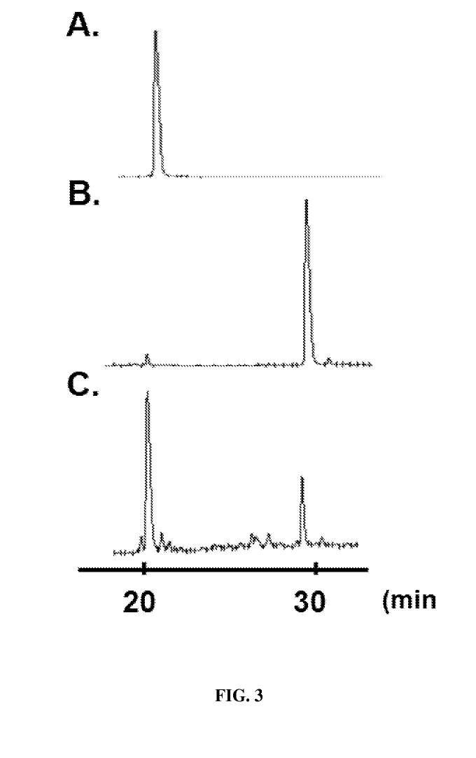

In order to determine the un-caging ability of the targeter (e.g., the peptide GGGGY(DMNB)IGSR—NH₂ (SEQ ID NO: 2)) leading to its transformation to the active state, solutions of non-caged peptide (GGGGYIGSR-NH₂) (SEQ ID NO: 2), or caged peptides subjected or not subjected to 1 min of illumination were evaluated by HPLC-UV. Retention time in the column of the non-caged targeter was ˜20 min (FIG. 3A) while that of the non-illuminated caged targeter was longer (˜30 min, FIG. 3B) due to the caging group existence which increases the hydrophobicity of the peptide. Ten seconds post illumination a shift in the retention time had occurred and the peptide had exited the column after ˜20 min (FIG. 3C). These results indicate that a quick release of the caging group from the peptide had occurred, leading to its rapid activation and preparation for efficient cell binding.

Next the amine terminated caged peptide/targeter was conjugated to the surface of carboxyl-terminated polystyrene nanoparticles using 1-ethyl-3-(3-dimethylaminopropyl) carbodiimide (EDC) and sulfo-N-hydroxysuccinimide (NHS) activation chemistry.

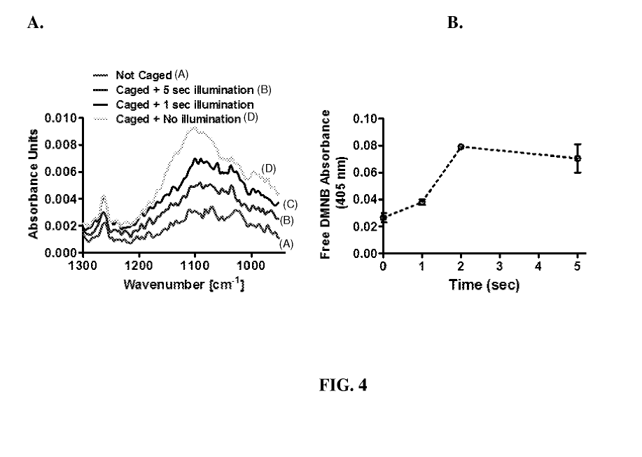

The caged nanoparticles have a broad peak at ˜1100 cm⁻¹ (consistent with ether stretch) by FTIR. In order to demonstrate a quick un-caging time of the DMNB, nanoparticles conjugated with the caged targeter were analyzed after illumination for 1 (C) and 5 sec (B). Non-caged targeter IR spectra served as control (A). Results reveal the disappearance of the ether bond from the targeter-conjugated nanoparticles after 5 sec (FIG. 4A) suggesting quick targeter activation. To further assess the amount of caging groups released to the media from the conjugated nanoparticles, the absorbance of DMNB by spectrophotometer was measured after illuminating the particles for 1, 2 and 5 sec (FIG. 4B). The released DMNB was then compared to a calibration curve of free DMNB and the ratio between the obtained value and the known available carboxylic acid number on the particles had suggested ˜85% DMNB release. Since each nanoparticle was conjugated to ˜5000 targeter molecules, statistically the amount of activated targeters will be sufficient for activation of every particle subjected to illumination and for cell binding. Furthermore, since activation of the targeter is concentration dependent (e.g., higher particle density may hinder light from passing to all particles), in vivo, the particles will be more abundant and the light will be able to promote faster activation of the particles circulating through the light beam.

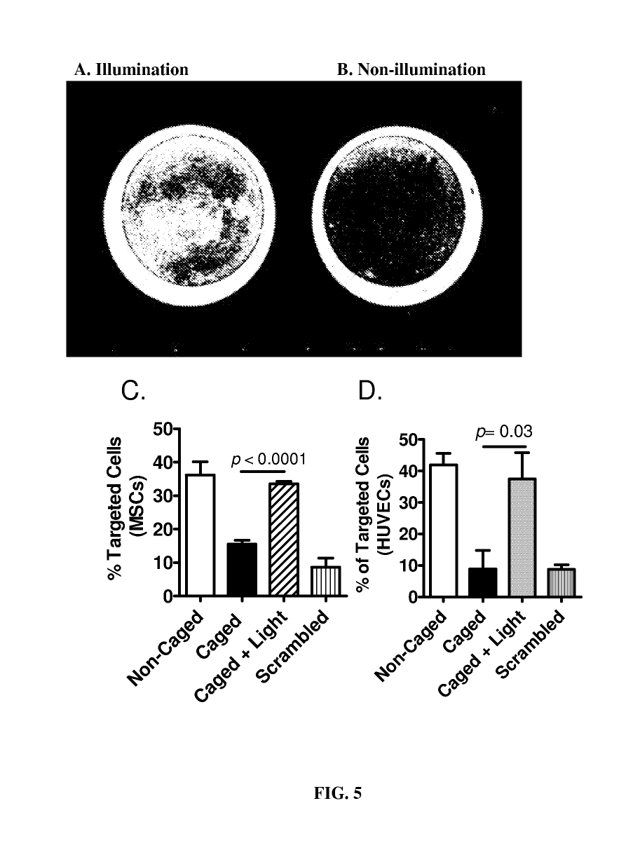

After ensuring that the nanoparticles are activated upon illumination, the ability of the caged particles to adhere to cells when exposed to light was evaluated. As a first step, human umbilical vein endothelial cells (HUVECs) were seeded in 60 mm culture dishes. Twenty four hours later, the culture medium was replaced with media containing caged nanoparticle and the dishes were illuminated for 1 min. After 15 min of incubation, the dishes were carefully washed and placed under UV light transilluminator for visualization of cell targeting. Non-illuminated cultures served as control. While the nanoparticles (appear in white) in the dishes subjected to light adhered to the cells and clearly covered most of the culture dish area (FIG. 5A) the non-illuminated cultures were mostly without nanoparticles, with some nanoparticles adhering at the edges (FIG. 5B).

After qualitatively having confirmed the feasibility of particle adherence to the HUVECs, the potency of the illumination in promoting cell targeting was quantitatively assessed. Since this targeting system was not designed to selectively distinguish between different cell types but to target any cell or tissue in the presence of light, the targeting experiments were performed with HUVECs and mesenchymal stem cells (MSCs), two cell types which may represent a broader population of cells expressing integrin 131. These two cell types were chosen since HUVECs represent cells comprising blood vessels and MSCs represent stromal cells present in connective tissues. Together and separately these cell categories are found in every tissue and organ in the body. Thus, the potential of the nanoparticles to target and bind to specific areas, tissues and organs in the body is only photo-dependent.

The cells (HUVECs and MSCs) were seeded in culture slides and allowed to recover. Twenty four hours after seeding, the culture media was replaced with media containing 10 μg fluorescent caged particles, illuminated for 1 min, incubated for 30 min and then immediately washed, fixed and stained. The number of cells which were targeted by the nanoparticles was counted under the microscope and divided by the total cell number. As control, either caged nanoparticles not exposed to light, nanoparticles conjugated with a scrambled peptide as a targeter which were illuminated or with un-caged YIGSR-NH₂ conjugated nanoparticles (positive control) were used.

In both cell type cultures, HUVECs and MSCs, the percentage of particle attachment after illumination was significantly higher compared to the non-illuminated cultures (p=0.03 and p<0.0001 for HUVECs (FIG. 5C) and MSCs (FIG. 5D) cultures, respectively. Furthermore, binding of caged nanoparticles exposed to light to the cells was at the same level as the positive control, particles conjugated to un-caged targeters (p=0.53 in MSCs), indicating efficient release of the caging group and efficient activation of the nanoparticles.

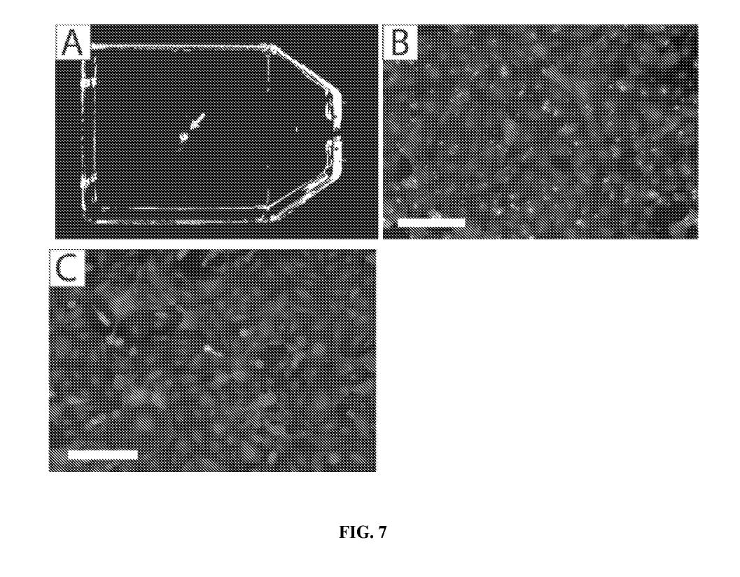

Finally, the spatial targeting ability of the caged particles was assessed. HUVECs were cultivated with caged nanoparticles in a 25 cm² T flask. The flask was covered with a mask allowing light penetration only in its center (d=1 mm) and was placed in the dark under an inverted microscope. Since every movement of the flasks after the particles are activated results in nanoparticle shift and attachment to areas not subjected to light, the flasks were fixed in the dark for 10 min prior to exposure to the microscope focused light beam for 1 min. To minimize particle diffusion distance, the cultures were incubated for this short period with a high concentration of caged nanoparticles (200 μg/mL). The nanoparticles were activated and attached to cells where light was introduced. The particles were arranged in a circle shape corresponding to the shape of the mask. Although the mask cleft diameter was only 1 mm, the targeted cells were located in a diameter of ˜6 mm probably due to nanoparticle diffusion after activation or scattering of the light beam. More than 94% of the cells at the center of the light beam were targeted by the particles (FIG. 7B) while almost no cell targeting was seen at the area not exposed to light (FIG. 7C).

In conclusion, described herein is a targeting system capable of binding to cells selectively upon illumination. Since these cells are present in every tissue in the body, this targeting system may be feasibly used for targeting diseased tissues without taking into consideration expression of specific markers.

### REFERENCES

1. Peer, D.; Karp, J. M.; Hong, S.; Farokhzad, O. C.; Margalit, R.; Langer, R. Nature Nanotechnology 2007, 2, (12), 751-60.
2. Duncan, R. Nat Rev Drug Discov 2003, 2, (5), 347-60.
3. Mallidi, S.; Larson, T.; Tam, J.; Joshi, P. P.; Karpiouk, A.; Sokolov, K.; Emelianov, S. Nano Letters 2009, 9, (8), 2825-31.
4. Bagalkot, V.; Zhang, L.; Levy-Nissenbaum, E.; Jon, S.; Kantoff, P. W.; Langer, R.; Farokhzad, O. C. Nano Lett 2007, 7, (10), 3065-70.
5. Farokhzad, O. C.; Cheng, J.; Teply, B. A.; Sherifi, I.; Jon, S.; Kantoff, P. W.; Richie, J. P.; Langer, R. Proc Natl Acad Sci USA 2006, 103, (16), 6315-20.
6. von Mehren, M.; Adams, G. P.; Weiner, L. M. Annu Rev Med 2003, 54, 343-69.
7. Dvorak, H. F.; Nagy, J. A.; Dvorak, A. M. Cancer Cells 1991, 3, (3), 77-85.
8. Bisby, R. H.; Mead, C.; Morgan, C. G. FEBS Lett 1999, 463, (1-2), 165-8.
9. Manyak, M. J.; Russo, A.; Smith, P. D.; Glatstein, E. J Clin Oncol 1988, 6, (2), 380-91.
10. Kohane, D. S. Biotechnol Bioeng 2007, 96, (2), 203-9.
11. Langer, R.; Tirrell, D. A. Nature 2004, 428, (6982), 487-92.
12. Epstein-Barash, H.; Shichor, I.; Kwon, A. H.; Hall, S.; Lawlor, M. W.; Langer, R.; Kohane, D. S. Proc Natl Acad Sci USA 2009, 106, (17), 7125-30.
13. Ellis-Davies, G. C. Nat Methods 2007, 4, (8), 619-28.
14. Graf, J.; Ogle, R. C.; Robey, F. A.; Sasaki, M.; Martin, G. R.; Yamada, Y.; Kleinman, H. K. Biochemistry 1987, 26, (22), 6896-900.
15. Grant, D. S.; Tashiro, K.; Segui-Real, B.; Yamada, Y.; Martin, G. R.; Kleinman, H. K. Cell 1989, 58, (5), 933-43.
16. Graf, J.; Iwamoto, Y.; Sasaki, M.; Martin, G. R.; Kleinman, H. K.; Robey, F. A.; Yamada, Y. Cell 1987, 48, (6), 989-96.
17. Wang, Y. G.; Samarel, A. M.; Lipsius, S. L. J Physiol 2000, 526 Pt 1, 57-68.
18. Maeda, T.; Titani, K.; Sekiguchi, K. J Biochem 1994, 115, (2), 182-9.
19. Rhee, H.; Lee, J. S.; Lee, J.; Joo, C.; Han, H.; Cho, M. J Phys Chem B 2008, 112, (7), 2128-35.
20. Karpen, J. W.; Zimmerman, A. L.; Stryer, L.; Baylor, D. A. Proc Natl Acad Sci U S A 1988, 85, (4), 1287-91.
21. Barbash, I. M.; Chouraqui, P.; Baron, J.; Feinberg, M. S.; Etzion, S.; Tessone, A.; Miller, L.; Guetta, E.; Zipori, D.; Kedes, L. H.; Kloner, R. A.; Leor, J. Circulation 2003, 108, (7), 863-8.
22. Dvir, T.; Benishti, N.; Shachar, M.; Cohen, S. Tissue Eng 2006, 12, (10), 2843-52.
23. Dvir, T.; Levy, O.; Shachar, M.; Granot, Y.; Cohen, S. Tissue Eng 2007, 13, (9), 2185-93.

This invention is not limited in its application to the details of construction and the arrangement of components set forth in the above description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

We claim:

1-10. (canceled)

11. A method for targeted delivery of a substance to predefined cells or tissues in an individual comprising:
(a) administering to an individual in need thereof a composition comprising particles containing an amount of a substance to be delivered to the individual, wherein a targeting ligand inactivated by caging using a photo-removable protecting group is attached to the surface of the particles;
(b) selectively irradiating predefined cells or tissues in the individual to activate the inactive ligand in the irradiated predefined cells or tissues by removal of the protecting group, wherein the active ligand is capable of binding along with the attached particles to an anti-ligand present on the predefined cells or tissues leading to the targeted delivery of the substance to the individual.

12. The method of claim 11, wherein the ligand comprises peptides, antibodies, aptamers.

13. The method of claim 12, wherein the peptide comprises a RGD or YIGSR (SEQ ID NO: 1) motif.

14. The method of claim 11, wherein the photo-removable protecting group is selected from a group consisting of 2-nitobenzyl, benzoin esters, N-acyl-7-nitindolines, meta-phenol, phenacyls and derivatives thereof.

15. The method of claim 14, wherein the photo-removable protecting group is a 4,5-dimethoxy-2-nitrobenzyl (DMNB) or a derivative thereof.

16. (canceled)

17. The method of claim 11, wherein two or more different targeting ligands are attached to the surface of the particles.

18. The method of claim 17, wherein at least one of the targeting ligands is tissue specific or cell-type specific.

19-20. (canceled)

21. A method for targeted delivery of a substance to predefined cells or tissues in an individual comprising:
(a) administering to an individual in need thereof a composition comprising particles containing an amount of a substance to be delivered to the individual, wherein a targeting peptide inactivated by caging using a photo-removable protecting group is attached to the surface of the particles;
(b) selectively irradiating predefined cells or tissues in the individual to activate the inactive peptide in the irradiated predefined cells or tissues by removal of the protecting group, wherein the active peptide is capable of binding along with the attached particles to integrins present on the predefined cells or tissues leading to the targeted delivery of the substance to the individual.

22. The method of claim 21, wherein the peptide comprises a RGD or YIGSR (SEQ ID NO: 1) motif.

23. The method of claim 21, wherein a second targeting ligand is attached to the surface of the particles.

24. The method of claim 23, wherein the second targeting ligand is tissue specific.

25. A composition comprising:
a plurality of particles, wherein each particle is capable of carrying an amount of a substance to be delivered to an individual, wherein a targeting ligand inactivated by caging using a photo-removable protecting group is attached to the surface of the particles, wherein the inactive ligand is activated by removal of the protecting group by irradiation of the composition, and wherein the active ligand is capable of binding an anti-ligand.

26. The composition of claim 25, wherein the ligand comprises peptides, antibodies, aptamers.

27. The composition of claim 26, wherein the peptide comprises a RGD or YIGSR (SEQ ID NO: 1) motif.

28. The composition of claim 25, wherein the photo-removable protecting group is selected from a group consisting of 2-nitobenzyl, benzoin esters, N-acyl-7-nitindolines, meta-phenol, phenacyls and derivatives thereof.

29. The composition of claim 28, wherein the photo-removable protecting group is a 4,5-dimethoxy-2-nitrobenzyl (DMNB) or a derivative thereof.

30. The composition of claim 25, wherein the photo-removable protecting group is covalently attached to the ligand.

31. The composition of claim 25, wherein two or more different targeting ligands are attached to the surface of the particles.

32. The composition of claim 31, wherein at least one of the targeting ligands is tissue specific or cell-type specific.

33-34. (canceled)

35. The composition of claim 25, wherein each particle is carrying an amount of a substance to be delivered.
